(19) United States
(12) Patent Application Publication
(10) Pub. No.: US 2026/0174844 A1
(43) Pub. Date: Jun. 25, 2026

(54) ADMINISTRATION OF VACCINES TO SITES OF TEMPORALLY INDUCED CELL-MEDIATED HYPERSENSITIVITY REACTIONS, TO FACILITATE THE DEVELOPMENT OF PROTECTIVE SENSITIZATION AGAINST INFECTIOUS DISEASES AND CANCERS

(71) Applicants: Robert E. COIFMAN, Millville, NJ (US); Catherine F. YANG, Elk Grove, CA (US)

(72) Inventors: Robert E. COIFMAN, Millville, NJ (US); Catherine F. YANG, Elk Grove, CA (US)

(21) Appl. No.: 19/536,686

(22) Filed: Feb. 11, 2026

Related U.S. Application Data
(63) Continuation-in-part of application No. 17/394,506, filed on Aug. 5, 2021, now abandoned.
(60) Provisional application No. 63/063,072, filed on Aug. 7, 2020.

(51) Int. Cl. A61K 39/35; A61K 39/00

(52) U.S. Cl. CPC A61K 39/35; A61K 2039/542; A61K 2039/544; A61K 2039/545; A61K 2039/572

(57) ABSTRACT

Provided are methods of biasing a response of a vaccine recipient's immune system toward protective sensitization. Methods include administering an agent or substance to the site of a subject of a temporally interacting cell-mediated allergic reaction to enhance the resulting level of protective sensitization.

### FIELD

The present disclosure relates to the deliberate induction of a cell-mediated hypersensitivity reaction, clinically known as allergic contact dermatitis, to modify the biology of the skin to which a vaccine is applied, to bias the response to that vaccine toward stronger protective immunity.

### RELATED APPLICATIONS

This application is a continuation-in-part application of U.S. patent application Ser. No. 17/394,506, filed on Aug. 5, 2021, which claims the benefit of U.S. Provisional Application 63/063,072, filed on Aug. 7, 2020, the contents of both of which are incorporated herein by reference in their entireties.

### BACKGROUND

A vaccine is defined by the CDC as “A product that stimulates a person's immune system to produce immunity to a specific disease, protecting the person from that disease.” Vaccines act by either reinforcing an existing state of immune system responsiveness or triggering immunomodulation from one state of responsiveness to another. Vaccines to protect patients from allergic diseases are designed to induce immunomodulation from pathological states of sensitization to immunological tolerance. Vaccines to protect against infectious diseases can either induce immunomodulation from immunological naiveté to protective sensitization or boost or enhance an existing state of protective sensitization. Vaccines to protect against cancer are designed to induce immunomodulation from tolerance of a patient's own cancer cells to a state of protective immunity.

### BRIEF SUMMARY

Methods are provided in which an inflammatory response is induced to modify one or more of the dendritic cell population in tissue of a vaccine recipient to which a vaccine is administered and the cytokine milieu in that tissue and/or the draining lymph nodes to which antigens picked up by dendritic antigen presenting cells will be taken for processing. Pro-inflammatory changes in the microenvironments in which the antigens present in the vaccine will be processed bias the response in the direction of protective sensitization. Thus, methods include biasing a response of a vaccine recipient's immune system toward protective sensitization. Among the factors determining the immune response of the recipient of a vaccine are the populations of dendritic antigen-presenting cells and the cytokine milieu present in the tissue to which the vaccine is introduced. The cytokine mix in the lymph node in which the antigens present in the vaccine are presented to naïve T-cells can also affect the pathway along which those naïve T-cells differentiate following costimulation.

The present application is directed to methods that include administering vaccines against infectious diseases and cancers in association with natural or induced cutaneous cell-mediated allergic reactions as a way to enhance the resulting protective sensitization.

### BRIEF DESCRIPTION OF THE FIGURE

FIG. 1 is a graphic representation from Teunissen et al. of the function of the adaptive immune system in the skin and mucosal membranes that have evolved with similar exposure to the microbiota of the world.

### DETAILED DESCRIPTION

While most vaccines are currently given by mouth or subcutaneously or by intramuscular injection, the first effective vaccine against an infectious disease (Edward Jenner's vaccine against smallpox) was given by multiple punctures through a drop of active vaccine into the epidermis and dermis. The skin may be a target of interest for the administration of vaccines to protect against both infectious and neoplastic diseases to exploit the presence and organization of cells and cell types within the skin whose ability to facilitate protective sensitization is an evolutionary task of our skin to protect those of our tissues that live inside it. The mucosa of the upper respiratory tract and particularly of the nasal cavity have the same evolutionary role and are also target tissues for different embodiments of the methods of the present application.

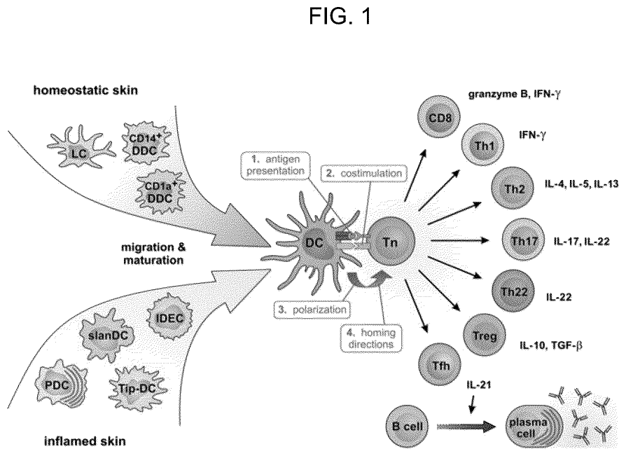

Since the publication of 2012 M. B. M. Teunissen edited vol. 351 of Springer's Current Topics in Microbiology and Immunology on Intradermal Immunization (“Teunissen”), there have been quantitative advances in the field in the form of additional and improved applications of the principles and methods described in that volume but basic understanding of the steps and mechanisms of immunomodulation remains as illustrated in FIG. 1 adapted from that volume.

As used herein, “Immunomodulation” is the process of inducing a change in the immune response to a particular substance.

As used herein, an “antigen” is a molecule, typically a protein, peptide, polysaccharide, lipid or nucleic acid, that can be presented in a manner in which it can be recognized by the immune system and trigger a specific immune response.

As used herein, an “epitope” is the specific part of an antigen that is presented to a naïve T-cell in the cortex of a lymph node to induce or reinforce or modify a previously induced immune response. The resulting immune response will be directed at that part of the antigen.

As used herein, a “vaccine” is a pharmaceutical formulation intended for administration to a human or animal to achieve immunomodulation in a specified direction. A traditional vaccine will contain one or more antigens. Alternative vaccine technologies have been developed that use either isolated epitopes or RNA that codes for variably sized fractions up to 100% of complete antigens

As used herein, an “inflammatory reaction” is the body's natural response to injury, infection, or harmful stimuli, involving immune cells, blood vessels, and molecular mediators to eliminate the cause and initiate healing. Inflammatory reactions can be caused by infections, physical injuries, chemical irritants, or immune system dysfunctions such as cell-mediated hypersensitivity reactions.

As used herein “cell-mediated hypersensitivity” is a reaction of the adaptive immune system that uses T-lymphocytes to recognize and destroy infected cells, such as those harboring viruses or fungi. It primarily involves the activation of cytotoxic T lymphocytes (CTLs), which kill infected host cells by triggering programmed cell death (apoptosis), and T helper cells, which coordinate the immune response and activate other immune cells like macrophages. This type of immunity is crucial for fighting intracellular pathogens that antibodies cannot reach.

Cell-mediated hypersensitivity to the tumor-specific antigens present on every cancer cell are a way our bodies eliminate most mutant clones that could become cancers before they can become established. Every clinically identifiable cancer that becomes clinically apparent reflects a failure of the host's immune system to achieve a sufficient level of protective sensitization.

It is contemplated that there may be therapeutic changes other than changes in dendritic cell population and cytokine milieu. As used herein, the term “temporal”, or “temporally” interacting is meant to include sufficiently related by time of administration that the induced cell-mediated reaction alters the immune response of the host compared to that observed or expected to the same vaccine in the absence of the induced cell-mediated reaction.

The presence and function of the immune system is qualitatively the same in mucosal surface tissues as it is in the skin. To minimize the need to be repetitive, as used herein, any reference in this document to the “skin” will also apply to mucosal surface tissues that are exposed to the owner's microbiome and the microbiota of the environment These include the conjunctiva, which are not typically used as a site of vaccine delivery because of proximity to the eye, and the upper respiratory tract, which may be seen as a set of target tissues for the application of the present application, and the mouth.

FIG. 1 is a graphic representation from Teunissen et al. of the function of the adaptive immune system in the skin and mucosal membranes that have evolved with similar exposure to the microbiota of the world. A cutaneous cell-mediated allergic reaction creates a localized inflammatory dendritic cell and cytokine milieu, replicating many of the micro-environmental characteristics believed to facilitate protective sensitization as illustrated in FIG. 1. In FIG. 1. dendritic cells (DC) present in normal skin under normal conditions are shown at upper left. Additional dendritic cell populations induced and/or recruited by an inflammatory reaction, an example embodiment being a cell-mediated hypersensitivity reaction to a substance that may or may not be antigenically related. In the center of FIG. 1 is the tryst between a dendritic cell presenting antigen and a naive T-lymphocyte receiving it.

At the right of FIG. 1 are a number of pathways by which that T-lymphocyte could mature and function, depending on the type of dendritic cell and the micro-environmental chemical milieu in which this liaison takes place.

FIG. 1 shows DCs control the development of distinct T cell responses. After internalization of environmental antigens, cutaneous DCs migrate to the skin draining lymph node while undergoing a process of maturation to acquire the unique capacity to prime naive T cells (Tn). The different DC subsets in homeostatic skin and additional DC subsets in inflammatory conditions are indicated on the left site. The original antigenic stimulus and the local mediators from the neighboring cells at the site of infection are integrated by the DC into four signals (indicated in the numbered text boxes in the center of the page) that direct the development of the required type of effector T cell.

The factors illustrated on FIG. 1 other than the chemical nature of the antigen being passed from dendritic cell “DC” to naïve lymphocyte “Tn” in the center of the FIGURE, including the lineage of the dendritic cell that picks up each molecule of antigen and the cytokine milieu in both the tissue from which it came and the lymph node in which the handout takes place, can profoundly affect both the type and the intensity of the resulting immune response. The evolutionary survival value of being able to amplify protective sensitization in the face of an inflammatory stress makes it likely that certain stages of certain types of inflammatory reactions “C” with certain temporal and spatial relationships to certain types and formulations of certain vaccines “A” will yield sweet spots of dendritic cell population and cytokine milieu surrounding the site of vaccine delivery and in the draining lymph nodes of that tissue to bias the resulting immune response toward protective sensitization compared to what it would be in the absence of said inflammatory reaction(s).

The conditioning inflammatory reaction “C” can be a cell-mediated hypersensitivity reaction to conditioning antigen “B”. The dendritic cell and cytokine milieu of a cell-mediated hypersensitivity reaction to one antigen is intuitively more likely than a non-specific inflammatory reaction to be a micro-environment that favors the development of that type of reaction to another, though intuition is not infallible.

Evolutionary tissue bias is a factor that may be used to provide a bias at baseline in a target direction Skin and mucosal surfaces exposed to the microbiological flora of the external environment have the evolutionarily role of facilitating protective sensitization to potential sources of infection. In internal tissues the primary evolutionary role of the immune system is the maintenance of tolerance to self. The main immunological task of the gastro-intestinal system is to facilitate a combination of local and systemic tolerance to potentially allergenic foods so bodies can use them as sources of nutrition. The only successful oral vaccines for infectious disease are live vaccines that replicate in the host, for which the oral route is simply a convenient method of administration. The oral mucosa is not believed to have been studied as a site for vaccine administration.

Skin is the largest body issue evolutionarily predisposed to the development of protective sensitization. The nasal mucosa is a particularly favorable site as it's the natural portal of entry for many respiratory viral infections. The oral mucosa is another body surface tissue with an evolutionary history of defending against the many of the microbiota of the environment. Inducing protective sensitization in the nasal mucosa will generate local as well as systemic immunity. The local immunity will reduce the likelihood of infection and the risk associated with current vaccines for SARS Co-V2, that the vaccines will prevent or reduce symptoms but allow infected individuals to become asymptomatic spreaders.

The skin, the nasal mucosa and for some embodiments the oral mucosa are amenable to Antigen Delivery by Precipitation (ADBP), as described for example, in U.S. Published Application 2020-0222531 A1 and U.S. Pat. No. 9,107,901, which are incorporated herein by reference, in which an effective treatment dose of a water-insoluble antigen dissolved in a small volume of a pharmaceutically acceptable water-miscible solvent is either injected or topically applied in a solvent that will carry it across the skin or mucosal barrier, will deposit particles within a volume of the recipient tissue in a size range for effective dendritic cell uptake. Embodiments of the present methods may include administration of the vaccine to a vaccine recipient by ADBP.

A conditioning inflammatory reaction “C” may be induced any of locally, systemically or in another location with drainage to the same set of lymph nodes.

For different combinations of vaccine “A”, the target tissue to which it will be delivered and the nature of the conditioning inflammatory reaction “C”, the administration of vaccine “A” can be any of contemporaneous with the induction of the conditioning inflammatory reaction or at any time interval before or after the induction of “C” that proves most effective for that particular combination of dose and formulation of vaccine “A”, target tissue of administration, and type and intensity of conditioning inflammatory reaction “C”.

The conditioning inflammatory reaction may be a cell-mediated hypersensitivity reaction to a conditioning antigen “B”.

To fit within the scope of the present application conditioning antigen “B” must have at least some antigenic difference from vaccine “A”, rather than booster dosing. Conditioning antigen “B” may or may not be partially cross-reactive with vaccine “A”.

In the present application, the purpose and effect of every covered intervention is to alter the cellular and/or humoral composition of either or both of what is depicted on the left hand side of FIG. 1, the microenvironment in which target antigen “B” is picked up by a dendritic antigen-presenting cell, and the middle of the FIGURE depicting the cytokine environment of the lymph node in which the direction and intensity of the immune response are determined, in pro-inflammatory directions.

A non-limiting target immune response based on current understanding is to promote the top two immune responses on the right hand side of the FIGURE, the generation of CD8 and Th1 lymphocyte responses. For different embodiments, different interventions “S” and different target antigens “B” there are likely to be different sweet spots in the time course of the inflammatory reactions initiated by those different interventions “S” to optimize temporal and spatial as well as dosing relationships with administration of target antigens “B” to maximize the desired effect. While it is helpful to have cytological evidence of change in a relevant micro-environment and a luxury to have data documenting a change in the distribution of cytokines, neither is necessary to confirm coverage by the present application. If it is hot, red, swollen, itchy or any combination or if it's known that higher doses of the same intervention “S” cause any of those signs or symptoms one can conclude that any changes in outcome associated with that intervention are the result of inflammation present at a microscopic if not at a gross scale following a lower dose than those associated with gross changes indicative of inflammation.

Embodiments of the present application provide methods of biasing a response of a patient's immune system toward protective sensitization to a target antigen “B” covered by a vaccine “B” to be administered cutaneously or intra-nasally, that include administering a conditioning antigen “A” to target tissue of the patient to induce a cell-mediated hypersensitivity reaction of scope to encompass and modify an immunological environment in the target tissue to which vaccine “B”, to which protective sensitization is desired, will be administered, and administering the vaccine “B” to the patient cutaneously or intranasally to the target tissue. In example embodiments, vaccine “B” includes a solution of a water-insoluble antigen in a pharmaceutically acceptable water-miscible solvent such that upon administering the vaccine “B” to the patient, the vaccine “B” is diluted by available water content of tissue of the patient and the antigen becomes insoluble and precipitates into dispersed individual particles in the tissue.

In example embodiments, the vaccine “B” is antigenically related to the conditioning antigen “A”. In example embodiments, the vaccine “B” is not antigenically related to the conditioning antigen “A”.

As used herein, the terms “patient”, “subject” and “vaccine recipient” are used interchangeably.

In example embodiments, the patient to whom vaccine “B” is administered has been previously exposed to an antigen “B” covered by vaccine “B”.

In example embodiments, the patient to whom vaccine “B” is administered has not been previously exposed to an antigen covered by vaccine “B”.

In example embodiments, an antigen “B” of the vaccine “B” is a tumor antigen of the patient and the patient is a cancer patient.

In example embodiments, the tumor antigen has been modified to increase its antigenicity.

In example embodiments, the patient does not have previous protective cell-mediated hypersensitivity to the target antigen “B”.

Embodiments of the present application includes methods of biasing an immune response in a patient to a vaccine “B” toward protective cell-mediated hypersensitivity that include administering a conditioning antigen “A” to the patient to induce a cell-mediated hypersensitivity reaction in the patient that alters an immunological environment in the patient at a location into which a vaccine “B” for a target antigen “B” will be administered, the altered immunological environment induced by said cell-mediated hypersensitivity reaction to antigen “A” altering a signal vaccine “B” sends to an antigen response mechanism of the patient's immune system. In example embodiments, methods further include administering the vaccine “B” for the target antigen “B” to the patient. Vaccine “B” may include a solution of a water-insoluble antigen in a pharmaceutically acceptable water-miscible solvent such that upon administering the vaccine “B” to the patient, the vaccine “B” is diluted by available water content of tissue of the patient and the antigen “B” becomes insoluble and precipitates into dispersed individual particles in the tissue.

According to example embodiments, a patient does not have previous protective cell-mediated hypersensitivity to the target antigen “B”.

Non-limiting methods provided herein include methods of enhancing protective cell-mediated hypersensitivity in a patient induced by a locally administered vaccine “B” for a target antigen “B”, that include inducing a local cell-mediated hypersensitivity reaction in the patient by administering a conditioning antigen “A” to the patient; and administering the vaccine “B” for target antigen “B” to the patient, wherein a dose of conditioning antigen “A” used to induce a conditioning cell-mediated hypersensitivity reaction is administered in an interval from 30 days before administration of a dose of vaccine “B” to 14 days after administration of a dose of vaccine “B” in which wherein the vaccine “B” for the target antigen “B” is administered at a proximate site to the site of the cell-mediated hypersensitivity reaction to reflect histological or microenvironmental chemical changes caused by the cell-mediated hypersensitivity reaction or at a site drained by the same regional lymph nodes, and wherein the vaccine “B” includes a solution of a water-insoluble antigen in a pharmaceutically acceptable water-miscible solvent such that administering the vaccine for the target antigen to the subject, the vaccine “B” is diluted by available water content of tissue of the patient and the antigen becomes insoluble and precipitates into dispersed individual particles in the tissue.

In example methods, the vaccine “B” may be administered to a patient, for example by injection, of an effective amount of the vaccine “B” to a same site of the patient as the conditioning antigen “A”. In example embodiments, vaccine “B” may be administered to a patient within a predetermined distance as administration of the conditioning antigen “A”, such as within 2 cm or 1 cm or less.

According to example embodiments, the vaccine “B” may be administered to a different site of the patient than a site of administration of conditioning antigen “A”.

According to further non-limiting example embodiments, methods are provided of biasing a response of a vaccine recipient's immune system toward protective sensitization by induction of an immunomodulating cell-mediated allergic reaction in a specified temporal relationship and a specified spatial relationship to administration of the vaccine to the recipient, in which the specified temporal relationship includes induction of the immunomodulating cell-mediated allergic reaction from 0 to 21 days before vaccine administration to 0 to 21 days after vaccine administration, and in which the specified spatial relationship requires vaccine administration at a proximate site close enough to the site of the immunomodulating cell-mediated allergic reaction to reflect histological or microenvironmental chemical changes caused by the immunomodulating cell-mediated allergic reaction or at a site with drained by the same regional lymph nodes. According to example embodiments, the specified temporal relationship may include induction of the immunomodulating cell-mediated allergic reaction from 0 to 14 days before vaccine administration to 0 to 14 days after vaccine administration. The administered antigen may alter at least one of dendritic cell populations and/or cytokine milieu of the vaccine recipient.

In example methods, the temporal relationship includes induction of the immunomodulating cell-mediated allergic reaction from 0 to 24 hours before vaccine administration to 0 to 24 hours after vaccine administration.

The specified spatial relationship may include vaccine administration at the same site of the immunomodulating cell-mediated allergic reaction.

Also provided are methods of enhancing protective immunity in a subject or patient induced by a vaccine including administering to the subject an agent cutaneously or intra-nasally to a site of the subject to induce a cell-mediated allergic reaction, at the cellular and micro-environmental chemical level at the site, and administering a vaccine to the subject, for which the subject's immune system processes in tissues affected by a cell-mediated allergic reaction, to the subject before during or after administration of the antigen to induce a cell-mediated allergic reaction.

In example embodiments, the antigen may induce a cell-mediated allergic reaction. The antigen to induce a cell-mediated allergic reaction may be an antigen provoking a surface cell-mediated allergic reaction.

In non-limiting examples, the subject has been previously sensitized to the antigen provoking the surface cell-mediated allergic reaction. In example embodiments, the subject has not been previously sensitized to the antigen provoke the cell-mediated allergic reaction.

In example methods, the antigen provoking the cell-mediated allergic reaction is antigenically related to the immunizing antigenic material.

In example methods, the antigen provoking the cell-mediated allergic reaction is not antigenically related to the immunizing antigenic material.

In example methods, the vaccine is a tumor antigen of a tumor of the patient.

In example methods, the tumor antigen is modified prior to administering the vaccine to the subject to increase antigenicity.

In example methods, the antigen is administered cutaneously, and the antigen inducing the cutaneous cell-mediated allergic reaction is administered prior to, concurrently with, or subsequent to administration of the vaccine whose protective sensitization the cutaneous cell-mediated allergic reaction is intended to enhance.

In example methods, the induced cell-mediated reaction is internal.

Also provided are methods of enhancing protective immunity in a subject induced by a locally administered vaccine, including inducing a local cell-mediated allergic reaction in the subject by administrating a sensitizing agent to the subject; and administering a vaccine to the subject, wherein said local cell-mediated allergic reaction is induced 0 to 24 hours before vaccine administration to 0 to 24 hours after vaccine administration. The administered sensitizing agent alters at least one of dendritic cell populations and/or cytokine milieu of the vaccine recipient, in which the administered sensitizing agent includes an antigen.

In example embodiments, the sensitizing agent is applied topically to skin or lining membranes of nose of the subject.

In example embodiments, the sensitizing agent is applied to the subject internally.

In example embodiments, the sensitizing agent is applied to the subject by injection.

In examples, the vaccine may be applied to a same site on the subject as the agent provoking the cell-mediated allergic reaction.

In non-limiting examples, the vaccine is applied to a different site on the subject than the agent provoking the cell-mediated allergic reaction.

Embodiments of the present application include the induction of a localized, in most applications mild, natural cell-mediated allergic reaction and exploiting the immunomodulatory effect of that cell-mediated allergic reaction will have to bias the host response to a vaccine in the direction of protective sensitization compared to what it would be without the induced cell-mediated allergic reaction. Immune reactions are not all or none but span a wide range of intensity. A goal of the present methods may be to achieve clinically effective immunomodulation from naivete (in infections disease) or tolerance (in cancer) to a protective combination of cell-mediated and/or humoral immunity with a minimum of morbidity from the procedure making it desirable to induce the mildest exogenous cell-mediated allergic reaction needed to predictably and reliably achieve the desired immunomodulation. Possible mechanisms of action of the present application include but are not limited to local generation and/or stimulation of the influx of sensitizing dendritic cell populations and the creation in response to the inducing cell-mediated reaction of a cytokine milieu more favorable to the development of protective sensitization. Future technologies might make it possible to elicit and monitor a localized delayed hypersensitivity reactions in internal tissues, but at this time visual evaluation is only practical on the skin and in the nose. Their natural exposure to the outside world of potential sources of infection has given both of these tissues an evolutionarily predisposition to the development of sensitization, which the induced cell-mediated allergic reaction of this application may enhance. In each case and for each tissue the purpose of the local induced delayed hypersensitivity reaction is to create the immunological micro-environmental changes depicted in FIG. 1 to favor and facilitate immunomodulation from naivete or tolerance to protective sensitization.

Embodiments of the present application include all uses in which the induced cell-mediated reaction alters either or both of the dendritic cell populations and the cytokine milieu with (for the dendritic cells) or in (for cytokine milieu) which the immune system of the host responds to the vaccine.

An example of cell-mediated allergic reactions of this type are allergic contact dermatitis to poison oak and poison ivy, which are highly cross-reactive with each other and able to sensitize ˜85% of Americans. According to example embodiments, it may be preferable to use non-natural sensitizers to which potential recipients would not have the broad range of prior level of sensitization resulting from variable natural exposure to poison oak and poison ivy. A historically used non-natural contact sensitizer was dinitrochlorobenzene (DNCB). It was used as a topical treatment for warts. It created a local cell-mediated allergic reaction in the micro-environment in which antigen-presenting host dendritic cells were exposed to virus-specific antigens at the margins of a benign, virus-induced tumor that does not have the skill set of many malignant tumors to evade local immune system recognition. The dendritic cells that have picked up tumor cell surface antigens present them to the naive T-lymphocytes illustrated in the center of FIG. 1 whether a DNCB-induced cell-mediated reaction is present or not, but in the absence of the immunomodulating cell-mediated allergic reaction the wart survives and thrives. When the cell-mediated DNCB reaction is present, it biases the resulting immune response sufficiently in the direction of protective sensitization to generate a reaction that eliminates the tumor. The outcome is the same shifting of immune system response toward protective sensitization that the present application is designed to replicates for vaccines.

The induction of a natural cell-mediated immune reaction to a non-natural universal sensitizer to which the vast majority of the population has not been previously exposed would be the most practical way to develop a standardized protocol for the implementation of this application. Those (patients or recipients) with genetically normal cell-mediated immunologic reactivity could be expected to respond, and in most cases, to respond to a standardized dose, even if their genetically normal cell-mediated immune system was immunosuppressed. A goal of these embodiments of the present application is to achieve the same boost of protective immunity to an exogenously administered vaccine that direct application of DNCB achieves for a common wart. For immunotherapy of cancer the exogenously administered vaccine could be made from the patient's own tumor antigens either with or without intermediate modification to increase their antigenicity.

A concurrent or temporally proximate induced cell-mediated reaction in a location distant from a site of vaccine administration could potentially induce micro-environmental changes at a remote vaccine administration site that could induce protective immunomodulation if it causes no detectable microscopic or chemical change in the microenvironment of the vaccine administration site. Applicant submits that it's possible that a concurrent or temporally proximate induced cell-mediated reaction could induce a systemic effect on vaccine response and that any such case of distant induction of protective sensitization without identifiable changes in injection site histology of chemistry would still fall within the scope of this application.

There may be applications of the present application for which a booster or recall cell-mediated immune response is more effective than an initial sensitization response at creating a dendritic cell and cytokine milieu favorable to protective sensitization. It will be technically easiest to standardize a recall challenge dose for a non-natural sensitizer to which almost the only individuals previously exposed will be those who have previously received the treatment of this application.

Elicitation of a recall reaction to a natural sensitizer such as poison ivy may be an effective way to apply the present application to a cancer patient so severely immunosuppressed as to be unable to mount a response to a sensitizer to which they had not been previously exposed. A problem with general use of poison ivy is that the topical dose needed to induce the same degree of recall reaction was observed to vary by a factor of 39,000. (Coifman R E, Yang C F, Tolerance to poison ivy following vaccine delivery by precipitation, Annals of Allergy, Asthma and Immunology 2019(March); 122:331-33).

DNCB and other non-natural universal contact sensitizers cause a plurality of changes in dendritic cell populations and cytokine milieu. Changes will be different for sensitizing and recall reactions and also depend on the intensity of the induced cell-mediated reaction and the time in the course of that reaction at which the vaccine is administered. The different cellular and micro-environmental chemical effects of the different elements of the response to induced cell-mediated allergic reactions will have different time courses. When DNCB is repeatedly painted on a wart the patient benefits from a cumulative net positive balance of its net therapeutic and anti-therapeutic effects.

Where the antigen to which one wants to induce protective sensitization is only administered at one or a small number of discrete points in time, however, the temporal relationship between administration of the sensitizing agent and the vaccine may be important. As different vaccines that work by different mechanisms may have different “sweet spots” in their dose and temporal relationships to dosing of the sensitizing agent, it may take systematic small scale trial and error guided by reaction site cytology to identify these “sweet spots” of dosage and timing, which is believed to be within the skill of those in the art to determine based on the present disclosure.

In embodiments of the present application, the substance inducing the cell-mediated reaction may be applied to the intended vaccine administration site immediately prior to vaccine administration or a specified time interval prior to vaccine administration that may be determined to be most efficacious for a particular combination of sensitizer and vaccine. There may be applications for which the patient is sensitized sufficiently prior to vaccine administration for the dendritic cell and cytokine milieu of the intended site of vaccine administration to return to normal but that the sensitizer (to which by this time the patient is already sensitized) is applied to the intended vaccine administration site any of before, concurrent with, or even a specified time after administration of the vaccine. It is unlikely but theoretically possible for an initial dose of a sensitizer to which the recipient does not already have cell-mediated immunity to achieve protective immunomodulation if it is not applied until after the vaccine is administered, but any such instance will still fall within the scope of this application.

This application is submitted in the era of COVID-19, when numerous candidate vaccines of numerous types are being fast-tracked to expedite the discovery of something that works. Many of these tracks can be bent in the direction of that goal by:
1) Exploring the effect of an induced local cell-mediated allergic reaction on the response to intracutaneously or intranasally administered COVID-19 vaccines (as well as other vaccines for infectious diseases and cancers) toward more vigorous protective sensitization.
2) Performing small scale comparative trials, beginning in animal models when available, of the dose and timing variables described herein. Therapeutic induced cell-mediated vaccine application site reactions could be initiated prior to, concurrent with, or possibly even subsequent to administration of the vaccine to which the desired outcome is protective sensitization.
3) Expect that the optimal relationship of dose and timing may be different for different types of vaccines and different particle sizes which may be taken up by dendritic cells by different mechanisms. (Xiang S D, Scholzen A, Minigo G, et al. Pathogen recognition and development of particulate vaccines: does size matter? Methods. 2006; 40:1e9.)

The present application includes methods of biasing a response of a vaccine recipient's immune system toward protective sensitization by induction of an immunomodulating cell-mediated allergic reaction in a specified temporal relationship and a specified spatial relationship to administration of the vaccine to the recipient, wherein the specified temporal relationship includes induction of the immunomodulating cell-mediated allergic reaction from 0 to 21 days before vaccine administration to 0 to 21 days after vaccine administration. According to example embodiments, the specified temporal relationship includes induction of the immunomodulating cell-mediated allergic reaction from 0 to 24 hours before vaccine administration to 0 to 24 hours after vaccine administration. Inducing an immunomodulating cell-mediated allergic reaction may include administering an antigen cutaneously or intra-nasally. The antigen may alter at least one of dendritic cell populations and/or cytokine milieu of the vaccine recipient.

The specified spatial relationship may require vaccine administration at the same site as the immunomodulating cell-mediated allergic reaction, at a proximate site close enough to reflect histological or microenvironmental chemical changes caused by the immunomodulating cell-mediated allergic reaction, at a site with no identifiable histological or microenvironmental chemical changes but drained by the same regional lymph nodes, or independent of body location.

Provided herein by way of non-limiting example embodiments are methods that include the induction of cutaneous cell-mediated allergic reactions to modulate the response of the immune system to vaccines administered to areas of skin affected by said induced cell-mediated allergic reactions. At the cellular and micro-environmental chemical level these reactions consist of evolving series of inflammatory and reparative changes in both cell populations and cytokine milieu. Many steps of these processes replicate micro-environmental conditions known or believed to be associated with the enhancement of protective sensitization. The present application relates to the use of a temporally or contemporaneously interacting cutaneous cell-mediated allergic reaction to a substance that is not necessarily antigenically related to the vaccine being given.

Non-limiting example embodiments of the various methods of this application include topical administration to a subject of a standardized sensitizing or recall dose of a non-natural universal sensitizer to the skin or nasal mucosa (standardized to maximize likelihood of efficacy with minimum risk of unnecessary severity) followed either immediately or at a prescribed subsequent interval by a topically penetrating, locally precipitating formulation of the vaccine.

In non-limiting embodiments, the agent provoking an intranasal or cutaneous cell-mediated allergic reaction can be applied topically but the vaccine applied by injection into the nasal mucosa, dermis or sub-dermal space.

According to non-limiting example embodiments, the subject may have been previously sensitized to the antigen provoking the surface cell-mediated allergic reaction. According to other non-limiting example embodiments, the subject has not been previously sensitized to the agent inducing the cell-mediated allergic reaction.

According to non-limiting example embodiments, the antigen inducing the enhancing cell-mediated allergic reaction is antigenically related to the immunizing antigenic material. In other embodiments, the antigen provoking the cell-mediated allergic reaction is not antigenically related to some component of the immunizing antigenic material.

According to non-limiting examples, the substance provoking the cutaneous cell-mediated allergic reaction is administered prior to administration of the vaccine whose protective sensitization that cutaneous cell-mediated allergic reaction is intended to enhance.

The agent provoking the cutaneous cell-mediated allergic reaction may be administered temporally or contemporaneously with the vaccine whose protective sensitization the cutaneous cell-mediated allergic reaction is intended to enhance.

According to example embodiments, the antigen is administered cutaneously and the agent inducing the cutaneous cell-mediated allergic reaction is administered prior to, subsequently or subsequent to administration of the vaccine whose protective sensitization the cutaneous cell-mediated allergic reaction is intended to enhance.

In example embodiments, the locally induced therapeutic immunomodulatory cell-mediated reaction is internal.

Also provided are methods of enhancing protective immunity in a subject induced by a vaccine, that include inducing a local cell-mediated allergic reaction in the subject by administrating a sensitizing agent to the subject; and administering a vaccine to the subject. The local cell-mediated allergic reaction may be induced from 0 to 21 days before vaccine administration to 0 to 21 days after vaccine administration or from 0 to 24 hours before vaccine administration to 0 to 24 hours after vaccine administration.

Also provided are methods of enhancing protective immunity in a subject induced by a vaccine, that include administering to the subject an agent cutaneously or intra-nasally to a site of the subject to induce a cell-mediated allergic reaction, at the cellular and micro-environmental chemical level at the site. Example methods also include administering a vaccine to the subject, for which the subject's immune system processes in tissues affected by a cell-mediated allergic reaction, to the subject before during or after administration of the antigen to induce a cell-mediated allergic reaction.

According to non-limiting example embodiments, a sensitizing agent may be applied topically to skin or lining membranes of nose of the subject. A sensitizing agent may be applied to the subject internally. According to example embodiments, the sensitizing agent is applied to the subject by injection.

In example embodiments, the vaccine may be applied to a same site on the patient (or subject) as the agent provoking the cell-mediated allergic reaction. In example embodiments, the vaccine may be applied to a different site on the patient than the agent provoking the cell-mediated allergic reaction, or it may be applied to a similar location on the patient, such as within 1 cm or 2 cm. The vaccine administration may be at a proximate site close enough to the site of the immunomodulating cell-mediated allergic reaction to reflect histological or microenvironmental chemical changes caused by the immunomodulating cell-mediated allergic reaction or at a site with drained by the same regional lymph nodes.

The present application is intended to encompass any application of the present methods in which the application sites of the agent provoking the reaction and the vaccine are or are not identical, whether the vaccine is injected beneath the site of application of a provoking agent to the skin or whether the two sites are far apart.

The present application includes use of the cellular and micro-environmental chemical changes of a local cell-mediated allergic reaction in a subject to enhance the protective immunity induced by a locally administered vaccine temporally or contemporaneously with said cell-mediated reaction. The sensitizing agent may be applied topically to skin or lining membranes of nose of the subject to induce the cell-mediated allergic reaction in the subject.

Also provided herein are methods of enhancing the protective immunity induced by a locally administered vaccine that includes administering an antigen to induce a cell-mediated hypersensitivity reaction, at the cellular and micro-environmental chemical level, and administering a vaccine to a location in a human or other multi-cellular animal recipient at which a delayed hypersensitivity reaction either has been induced, is concurrently being induced or will shortly be induced. According to example embodiments, sensitizing agent may be applied topically to skin or lining membranes of nose of the subject.

In non-limiting examples of the present methods, the antigen to induce a cell-mediated hypersensitivity reaction is an antigen provoking a cutaneous cell-mediated allergic reaction. In non-limiting examples, the subject may have been previously sensitized to the antigen provoking the cutaneous cell-mediated allergic reaction. In other examples, the subject has not been previously sensitized to the antigen provoking the cell-mediated allergic reaction.

According to further non-limiting examples, the antigen inducing the enhancing cell-mediated allergic reaction may be antigenically related to the immunizing antigenic material. In other examples, the antigen provoking the cell-mediated allergic reaction is not antigenically related to some component of the immunizing antigenic material.

In non-limiting example embodiments, the substance provoking the cutaneous cell-mediated allergic reaction is administered prior to administration of the vaccine whose protective sensitization that cutaneous cell-mediated allergic reaction is intended to enhance. In other examples, the agent provoking the cutaneous cell-mediated allergic reaction is administered temporally or contemporaneously (i.e., concurrently or one immediately after the other) as the vaccine whose protective sensitization the cutaneous cell-mediated allergic reaction is intended to enhance. In further examples, the agent provoking the cutaneous cell-mediated allergic reaction is administered either prior to or subsequent to administration of the vaccine whose protective sensitization that cutaneous cell-mediated allergic reaction is intended to enhance.

It is conceivable that changes in immune system responsiveness caused by an induced cell-mediated allergic reaction could have the same beneficial immunomodulatory effect for vaccines administered other than intracutaneously, topically to the nasal mucosa or topically to the skin in a vehicle such as dimethyl sulfoxide capable of transporting it into the skin, a non-limiting example being subcutaneous injection beneath skin in which s cell-mediated allergic reaction has been or is concurrently being induced.

Provided herein are methods that include biasing a response of a vaccine recipient's immune system toward protective sensitization by induction of a defined type and intensity of inflammatory reaction in specified temporal and spatial relationships to a specified dose, formulation, method of administration and administration site of a vaccine to a vaccine recipient. The vaccine recipient may be a mammal, such as a human, in need of the vaccine. The biasing of the response achieves changes in the dendritic cell and/or cytokine environment of the administration site and/or its draining lymph nodes, that can bias the response of the recipient toward the development of protective sensitization. In example embodiments, the response may be biased more strongly than if the inflammatory reaction had not been induced. An “Afferent Immunomodulatory Environment” may include all of these alternatives. In example embodiments, the interventions may alter the afferent immunomodulatory environment in a pro-inflammatory direction.

Non-limiting embodiments of the application may allow induction of the immunomodulating cell-mediated allergic reaction anywhere in the area of the body draining to the same regional lymph nodes or at any body location chosen to minimize the resulting inconvenience to the recipient, which would be the case for a systemic immunomodulatory effect.

In example embodiments, the inflammatory reaction may be a cell-mediated hypersensitivity reaction.

In example embodiments, the tissue of the vaccine recipient to which the vaccine is administered, is skin or mouth mucosa or the upper respiratory tract of the vaccine recipient.

In example embodiments, the biasing of the response of the vaccine recipient's immune system includes administering a dose of an inducer of a conditioning inflammatory reaction. The dose of the inducer of the conditioning inflammatory reaction and the specified dose of the vaccine may be administered within 30 days of each other, or within 15 days of each other or within 5 days of each other according to non-limiting example embodiments.

In example embodiments, the patient/subject to be treated may be one who does not have previous protective cell-mediated hypersensitivity to the target antigen prior to the present methods being performed.

Example methods may include administering a vaccine using precipitation methods that include administering the vaccine in a solution that includes pharmaceutically acceptable water miscible solvent, such as antigen delivery by precipitation.

Although the present disclosure has been described in example embodiments, additional modifications and variations would be apparent to those skilled in the art. It is therefore to be understood that the present disclosure herein may be practiced other than as specifically described. Thus, the present embodiments should be considered in all respects as illustrative and not restrictive. Accordingly, it is intended that such changes and modifications fall within the scope of the present disclosure as defined by the claims appended hereto.

We claim:

1. A method comprising
biasing a response of a vaccine recipient's immune system toward protective sensitization by induction of a defined type and intensity of inflammatory reaction in specified temporal and spatial relationships to a specified dose, formulation, method of administration and administration site of a vaccine to tissue of a vaccine recipient,
wherein the biasing of the response changes dendritic cell and/or cytokine environment of the administration site and/or draining lymph nodes, to bias the response of the vaccine recipient toward development of protective sensitization.

2. The method of claim 1, wherein the inflammatory reaction is a cell-mediated hypersensitivity reaction.

3. The method of claim 1, wherein the tissue of the vaccine recipient to which the vaccine is administered is skin or mouth mucosa or upper respiratory tract of the vaccine recipient.

4. The method of claim 2, wherein the tissue of the vaccine recipient to which the vaccine is administered is skin or mouth mucosa or upper respiratory tract of the vaccine recipient.

5. The method of claim 1, wherein the biasing of the response of the vaccine recipient's immune system comprises administering a dose of an inducer of a conditioning inflammatory reaction; and
wherein the dose of the inducer of the conditioning inflammatory reaction and the specified dose of the vaccine are administered within 30 days of each other.

6. The method of claim 2, wherein the biasing of the response of the vaccine recipient's immune system comprises administering a dose of an inducer of a conditioning inflammatory reaction; and
wherein the dose of the inducer of the conditioning inflammatory reaction and the specified dose of the vaccine are administered within 30 days of each other.

7. The method of claim 1, wherein the method of administration of the vaccine is antigen delivery by precipitation (ADBP).

8. The method of claim 2, wherein the method of administration of the vaccine is antigen delivery by precipitation (ADBP).
